(19) United States
(12) Patent Application Publication
(10) Pub. No.: US 2004/0028645 A1
(43) Pub. Date: Feb. 12, 2004

(54) Artificial tear composition adapted to be used with contact lenses

(76) Inventor: Masood Chowhan, Arlington, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/415,802

(22) PCT Filed: Dec. 13, 2001
(86) PCT No.: PCT/US01/48550

(51) Int. Cl. A61K 31/765

(52) U.S. Cl. 424/78.27

(57) ABSTRACT

An aqueous artificial tear/rewetting drop solution is described. The solution may be utilized as both an artificial tear for persons wearing contact lenses and as a contact lens rewetting drop. The solution is based on the use of a citric acid/sodium citrate buffer system to prevent binding or other complexation between cationic antimicrobial preservatives and soft contact lenses, thereby making it possible to apply the solution directly to contact lenses while the lenses are being worn.

### BACKGROUND OF THE INVENTION

The present invention is directed to artificial tear compositions that are formulated in a manner such as to also be capable of being utilized as a rewetting drop for contact lenses. The dual function of the solutions of the present invention requires the solution to not only be effective as artificial tears, but also to be compatible with contact lenses, particularly soft contact lenses.

Ophthalmic solutions are sometimes used by patients in an inappropriate manner. For example, patients sometimes apply inappropriate artificial tear products to the eyes while wearing their contact lenses, or soak the lenses in a solution that is not designed to be used for this purpose. This inappropriate use of products can be either accidental or intentional. But in either case, the use of products that are not designed for treating contact lenses can result in ocular irritation, due to the uptake of preservatives or other ingredients by the lenses. An artificial tear and rewetting drop product that is specifically designed for application to contact lenses while the lenses are being worn would therefore be highly desirable.

Various formulations have been previously described and marketed as artificial tear solutions. Various types of formulations for rewetting contact lenses have also been described and marketed previously. As utilized herein, the phrase “rewetting drop” refers to a solution that can be applied to a contact lens while it is being worn by a patient in order to lubricate the lens and re-hydrate the lens, thereby making the lens more comfortable.

The objective of the present invention is to provide an improved artificial tear/rewetting drop that is particularly useful in connection with soft contact lenses. More specifically, the principal objective of the present invention is to provide a novel solution that is capable of functioning effectively as an artificial tear and rewetting drop without any of the ingredients of the solution being adsorbed to the surfaces of soft contact lenses.

### SUMMARY OF THE INVENTION

The present invention is based on the use of a citric acid/sodium citrate buffering system to prevent complexation or other uptake of antimicrobial agents or other ingredients of the present artificial tear solution by soft contact lenses.

In the preferred embodiment of the present invention, the citric acid/sodium citrate buffer system is combined with several other ingredients to provide a fairly complex combination of ingredients which are carefully balanced so as to provide an effective tear replacement solution that is also capable of being utilized as a rewetting drop for soft contact lenses.

The use of a citric acid/sodium citrate buffer system is a critical element of the present invention. Specifically, this buffer system allows the artificial tear/rewetting drop solutions of the present invention to be applied directly to contact lenses, particularly soft contact lenses. Although this type of buffer system has been utilized previously in solutions utilized to disinfect contact lenses, it has not been used in an artificial tear product of the type described herein, that is, an artificial tear product that can be applied directly to contact lenses while the lenses are being worn and is also suitable to be used as a rewetting drop for contact lenses.

In addition to the citric acid/sodium citrate buffer system, the artificial tear/rewetting drop solutions of the present invention will also contain one or more demulcents to facilitate hydration of the eye and an ophthalmically acceptable antimicrobial preservative.

### DESCRIPTION OF PREFERRED EMBODIMENT

The artificial tear/rewetting drop of the present invention has been developed based on several criteria. A principal criterion was that the product must satisfy the preservative effectiveness standards of the United States Pharmacopoeia and Japanese Pharmacopoeia, as well as similar standards in other countries. The preservative effectiveness test or “PET” standards require products to have sufficient antimicrobial activity such that there is no risk of microbial contamination of the product during the period it is being utilized by patients.

Although the PET requirement is an important consideration for the safety of the patient, the use of antimicrobial agents to preserve the formulation from microbial contamination creates a problem. Specifically, the use of antimicrobial agents in solutions to be applied to contact lenses creates a risk that patients' eyes will become irritated due to the antimicrobial agent becoming bound to contact lenses, adsorbed to the surfaces of the lenses, absorbed within porous contact lens materials, or otherwise taken up by soft contact lenses. This potential problem, which is generally referred to herein as involving an “uptake” of the antimicrobial agent by the contact lens, is particularly significant in the event the product is to be applied to the contact lenses while being worn, rather than simply being utilized to rinse or wash the contact lenses when removed from the eyes.

A second major criterion for the solution of the present invention was the need to provide a solution that imitates the effect of natural tear fluid in lubricating and hydrating the cornea, without interfering with the comfort of contact lenses while being worn.

The above-stated objectives have been achieved by combining a citric acid/sodium citrate buffer system with demulcents and an ophthalmically acceptable, cationic antimicrobial preservative. As indicated above, the buffer system prevents uptake (i.e., binding, adsorption and/or absorption) of the cationic antimicrobial agent by soft contact lenses, and thereby eliminates the risk of ocular irritation when the artificial tear/rewetting drop is applied directly to the contact lenses.

The concentration of the citric acid/sodium citrate buffer system required for the above-described purpose will depend on various factors, such as the particular cationic antimicrobial agent selected and the concentration of antimicrobial agent being utilized. The concentration of the buffer system required to eliminate or significantly reduce uptake of the antimicrobial agent by contact lenses is referred to herein as “an effective amount”. The concentration of the buffer system will generally be in the range of from about 0.25 weight/volume percent (“w/v %”) to about 0.65 w/v %.

The demulcents utilized in the present invention include all of those known in the art of artificial tear products. The preferred demulcent agents are hydroxypropyl methylcellulose (“HPMC”), glycols and dextrans. However, various other agents known to provide a demulcent effect and facilitate hydration of the eye may also be utilized. These agents also provide a cushioning effect, thereby increasing the comfort of the contact lenses on the eyes.

The amount of demulcent required to facilitate hydration of the eye and provide a cushioning effect will vary depending on the particular demulcent selected. The concentration required for the foregoing purposes is referred to herein as “an effective amount”. The concentration will generally be in the range of from about 0.01 w/v % to about 5 w/v %. The preferred concentration ranges for particular demulcents are as follows:

| | |
|---|---|
| Demulcent | Concentration |

| | | |
|---|---|---|
| Carboxymethylcellulose sodium (CMC) | 0.2-2.5 | w/v % |
| Dextran 70 | 0.1 | w/v % |
| Gelatin | 0.01 | w/v % |
| Glycerin | 0.2-1 | w/v % |
| Hydroxyethyl cellulose (HEC) | 0.2-2.5 | w/v % |
| Hydroxypropyl methylcellulose (HPMC) | 0.2-2.5 | w/v % |
| Methylcellulose | 0.2-2.5 | w/v % |
| Polyethylene glycol 300 | 0.2-1 | w/v % |
| Polyethylene glycol 400 | 0.2-1 | w/v % |
| Polysorbate 80 | 0.2-1 | w/v % |
| Polyvinyl alcohol | 0.1-4 | w/v % |
| Povidone | 0.1-2 | w/v % |
| Propylene glycol | 0.2-1 | w/v % |

The above-described demulcents may be utilized either alone or in combination with one or more other demulcents. The combined use of HPMC and dextran 70 is particularly preferred.

The cationic antimicrobial preservatives utilized in the present invention are selected from the group consisting of polymeric quaternary ammonium compounds and polymeric biguanides. The preferred antimicrobial preservatives are polyquaternium-1 and polyhexamethylene biguanide (“PHMB”). Polyquaternium-1 is most preferred.

The cationic antimicrobial agents described above will be utilized in an amount effective to prevent the artificial tear/rewetting drop solutions of the present invention from becoming contaminated by microorganisms. Such an amount is referred to herein as a “preservative effective amount”.

The concentration of the antimicrobial agent will be dependent on the particular antimicrobial agent selected and the concentration of the citric acid/sodium citrate buffer system. The general concentration range will be 0.00001 to 0.01 w/v %. The preferred solutions of the present invention will contain polyquaternium-1 in at a concentration of from about 0.0001 to 0.005 w/v %.

The artificial tear/rewetting drop formulations of the present invention are aqueous solutions and are formulated to be isotonic, relative to human tear fluid. The solutions will generally have an osmolality in the range of from about 200 to 400 milliosmoles/kilogram water (“mOsm/kg”), preferably from about 280 mOsm/kg to about 320 mOsm/kg. The solutions are also formulated to have a physiologically compatible pH.

The preferred embodiment of the artificial tear/rewetting drop solution of the present invention is described in the following Example.

### EXAMPLE 1

### Formulation of Preferred Embodiment

Formulation

| Ingredient | Amount (w/v %) |
|---|---|
| Dextran 70 | 0.1 |
| HPMC (2910) | 0.3 |
| Boric acid | 0.2 |
| Citric acid, monohydrate | 0.02 1 |
| Sodium citrate | 0.56 |
| NaCl | 0.516 |
| Mannitol | 0.7 |
| Disodium EDTA | 0.05 |
| Polyquaternium-1 | 0.001 (plus 5% excess) |
| NaOH/HCl | q.s. pH 7.0 |
| Water | q.s. 100 |

II. Physical & Optical Parameters

| | |
|---|---|
| pH | 7.05 |
| Osmolality (mOsm/kg) | 289 |
| Refractive Index | 1.3359 |
| Surface tension | 45 |
| (dynes/centimeter) | |
| Viscosity (centipoise) | 9.0 |

The foregoing formulation was prepared by means of the procedures described below:

I. Preparation of a Receiving Vessel

Prepare a sterile receiving vessel with sterile filtration assembly attached and calibrate the compounding vessel.

Calibrate the compounding vessel to 100% of the final volume with purified water:

II. Preparation of 2% HPMC Stock Solution

1. Weigh out HPMC powder

2. Heat purified water (50% of batch volume) to boiling. Stop heating.

3. While stirring with a high-speed propeller mixer, add the HPMC powder slowly until it is uniformly dispersed.

4. Add cold purified water to 100% of batch volume.

5. Place the container in a refrigerator overnight.

6. Filter the HPMC stock solution through a 151 μm-polish filter. Collect the filtered 2% HPMC solution in a clean container.

7. Label and store the solution in refrigerator for further use.

III. Compounding Steps

1. Add 2% HPMC stock solution in the sterile receiving vessel (“Container I”), and autoclave HPMC in the vessel at 121° C. for 30 minutes.

2. Remove the vessel from autoclave. Allow stirring while cooling down to room temperature.

3. In another clean receiving vessel (“Container II”), add purified water (20-30° C.) up to 50% of the final batch volume.

4. Weigh and add the following ingredients and add to Container II:

a. boric acid

b. mannitol

c. sodium chloride

d. sodium citrate

e. citric acid

f. dextran 70

g. disodium edetate

h. polyquaternium-1

With continuous stirring, dissolve above chemicals until solution is clear.

5. Measure the pH of the salt solution in Container II. If necessary, adjust pH to 7.0 using 6N sodium hydroxide and/or 6N hydrochloric acid.

6. Filter the salt solution in Container II through 0.22-micron Millipore sterilizing filter(s) into the sterile receiving vessel (Container I).

7. Adjust to 100% of final volume with purified water (20-30° C.) and measure the final pH aseptically.

### EXAMPLE 2

### Preservative Effectiveness Testing

The artificial tear/rewetting drop formulation described in Example 1 above was tested to determine if it meets PET standards of the United States Pharmacopoeia and Japanese Pharmacopoeia. The formulation was tested in accordance with the procedures described in the United States Pharmacopoeia, 24^{th} Edition (2000), Section 51, pages 1809-1811. The preservative system of the solution was determined to be effective against gram positive (Staphylococcus aureus) and gram negative (Escherichia coli, Pseudomonas aeruginosa) bacteria, yeast (Candida albicans) and mold (Aspergillus niger). Two samples of the formulation were tested (i.e., Lot Number 1 and Lot Number 2). The test data are shown in Tables 1 and 2 below.

TABLE 1
Preservative Effectiveness Results - Lot No. 1
CFU/mL

| Test | | | | | |
|---|---|---|---|---|---|
| Organisms | Initial | 7 days | 14 days | 21 days | 28 days |
| S. aureus | 1.1 × 10⁶ | <10 | <10 | <10 | <10 |
| P. | 8.9 × 10⁵ | <10 | <10 | <10 | <10 |
| aeruginosa | | | | | |
| E.coli | 1.0 × 10⁶ | <10 | <10 | <10 | <10 |
| C. albicans | 9.6 × 10⁵ | 3.7 × 10⁵ | 6.1 × 10⁴ | 8.2 × 10³ | 7.6 × 10² |
| A. niger | 1.6 ×10⁶ | 1.2 × 10⁴ | 1.5 × 10³ | 1.3 × 10³ | 1.6 × 10³ |

TABLE 2
Preservative Effectiveness Results - Lot No.2
CFU/mL

| Test | | | | | |
|---|---|---|---|---|---|
| Organisms | Initial | 7 days | 14 days | 21 days | 28 days |
| S. aureus | 1.1 × 10⁶ | <10 | <10 | <10 | <10 |
| P. | 8.9 × 10⁵ | <10 | <10 | <10 | <10 |
| aeruginosa | | | | | |
| E. coli | 1.0 × 10⁶ | <10 | <10 | <10 | <10 |
| C. albicans | 9.6 × 10⁵ | 4.5 × 10⁵ | 4.1 × 10⁴ | 8.8 × 10³ | 2.6 × 10³ |
| A. niger | 1.6 × 10⁶ | 8.5 × 10³ | 1.5 × 10³ | 2.1 × 10³ | 1.4 × 10³ |

The results set forth in Tables 1 and 2 above demonstrate that the formulation satisfies the PET standards of the United States Pharmacopoeia. The level of antimicrobial activity exhibited by the formulation also satisfies the PET standards of the Japanese Pharmacopoeia. The levels of antimicrobial activity required to satisfy the foregoing standards are set for the in the following table:

PET Criteria
(Log Reduction)

| | Bacteria | Fungi |
|---|---|---|
| USP | Day 7 - 1 log; | Show stasis over entire test; |
| | Day 14 - 3 logs; | No greater than 0.5 log |
| | No decrease in log reduction after | decrease in log reduction |
| | Day 14 | |
| Japa- | Day 14 - 3 logs; | No decrease in log |
| nese | No decrease in log reduction from | reduction from initial count |
| Pharma- | day 14 through day 28 | at 14 and 28 days |
| copoeia | | |

We/I claim:

1. An isotonic, aqueous solution suitable to be used as both an artificial tear for persons wearing contact lenses and as a rewetting drop for soft contact lenses, comprising:
a demulcent in an amount sufficient to provide a moisturizing and lubricating effect when the solution is applied to the eyes;
an ophthalmically acceptable cationic antimicrobial agent in an amount sufficient to prevent microbial contamination of the solution prior to use;
a citric acid/sodium citrate buffer system in an amount sufficient to prevent uptake of said antimicrobial agent by a soft contact lens when the solution is applied to the lens; and
water.

2. An artificial tear/rewetting drop solution according to claim 1, wherein the antimicrobial agent is selected from the group consisting of polymeric quaternary ammonium compounds and polymeric biguanides.

3. An artificial tear/rewetting drop solution according to claim 2, wherein the antimicrobial agent is selected from the group consisting of polyquaternium-1 and PHMB.

4. An artificial tear/rewetting drop solution according to claim 3, wherein the antimicrobial agent comprises polyquaternium-1.

5. An artificial tear/rewetting drop solution according to claim 4, wherein the demulcent is selected from the group consisting of cellulose derivatives, glycols and dextrans.

6. An artificial tear/rewetting drop solution according to claim 5, wherein the demulcent comprises a combination of a cellulose derivative and a dextran.

7. An artificial tear/rewetting drop solution according to claim 6, wherein the demulcent comprises a combination of dextran 70 and HPMC.

8. An aqueous solution suitable to be used as both an artificial tear by persons wearing contact lenses and as a rewetting drop for soft contact lenses, said solution having the following formula, wherein all amounts are expressed as weight/volume percent:
